(19) United States
(12) Patent Application Publication
(10) Pub. No.: US 2017/0020793 A1
(43) Pub. Date: Jan. 26, 2017

(54) PERSONAL WASH COMPOSITION

(71) Applicant: Conopco, Inc., d/b/a UNILEVER, Englewood Cliffs, NJ (US)

(72) Inventors: Andrew Richard AVERY, Ellesmere Port (GB); Arijit BERA, South 24 Parganas (IN); Kelvin Brian DICKINSON, Bebington, Wirral (GB); Richa Sureshchand GOYAL, Bangalore (IN); Emma Louise JARVIS, Tarporley (GB); Deeleep Kumar ROUT, Bangalore (IN); Prasanth Chennothu SOMAN NAIR, Bangalore (IN)

(73) Assignee: Conopco, Inc., d/b/a UNILEVER, Englewood Cliffs, NJ (US)

(21) Appl. No.: 15/038,741

(22) PCT Filed: Nov. 24, 2014
(86) PCT No.: PCT/EP2014/075383

§ 371 Date: May 24, 2016

(30) Foreign Application Priority Data
Dec. 3, 2013 (EP) 13195428.1

(51) Int. Cl. A61K 8/02; A61K 8/36; A61K 8/31; A61K 8/368; A61Q 5/02; A61K 8/34; A61K 8/365; A61K 8/20; A61K 8/46; A61K 8/44; A61Q 5/12; A61K 8/362

(52) U.S. Cl. CPC A61K 8/0291; A61Q 5/12; A61K 8/361; A61K 8/31; A61K 8/368; A61K 8/362; A61K 8/345; A61K 8/365; A61K 8/20; A61K 8/463; A61K 8/442; A61Q 5/02; A61K 2800/592

(57) ABSTRACT

The present invention relates to personal wash compositions, in particular shampoo compositions comprising beneficial oils. It has been a long felt need to solubilise beneficial oils such as mineral oil in wormlike micelles in surfactant systems. It is therefore an object of the present invention to provide a personal wash composition comprising functional levels of beneficial oil such as mineral oil. It has been found that mineral oil can be solubilised in wormlike micelles in a surfactant system with specific hydrophilic or lipophilic linker molecules.

### FIELD OF THE INVENTION

The present invention relates to personal wash compositions, in particular shampoo compositions comprising beneficial oils.

### BACKGROUND OF THE INVENTION

Shampoo compositions with benefit agents for conditioning, moisturizing, anti-dandruff, hair growth promoting etc are compositions which target cleaning along with one or more additional benefits. Such shampoo compositions with benefit agents are found to be preferred among consumers over the ones without any benefit agents. Benefit agents generally include beneficial oils like silicone oils, natural oils, mineral oils etc.

Shampoo compositions containing beneficial oils are known. However, such compositions contain the oil at very low levels. Incorporation of beneficial oils at high or functional levels into an aqueous surfactant solution is not always possible only by the selection of an electrolyte as it would result in phase separation disrupting the desired microstructure and substantially reduces viscosity.

Surfactant molecules can self-assemble into various microstructures based on the composition and conditions in which they are present. It is important to maintain the desired microstructure in products like shampoo since it is the microstructure that determines the flow properties, viscosity etc of the product.

It is well known that in aqueous solutions of cationic surfactants, for example, CTAB, long wormlike micelles are formed upon addition of some salts, such as KBr and odium salicylate or upon mixture with anionic surfactants. Wormlike micelles are also formed in mixtures of cationic-anionic and ionic-nonionic surfactants among others.

It has been reported that oils induce a rod-sphere transition in surfactant micellar solutions, leading to a reduction in viscosity. Therefore, there is still a need to solubilise different kinds of oils in wormlike micelles in surfactant systems.

U.S. Pat. No. 6,667,044 B1 discloses microemulsion gels based on water in oil type microemulsions comprising an oil phase essentially composed of not easily volatile components and an aqueous phase containing one or more polyethoxylated W/O emulsifiers and/or one or more polypropoxylated W/O emulsifiers and/or one or more polyethoxylated and polypropoxylated W/O emulsifiers and/or one or more monoesters, diesters, polyesters of polyols as W/O emulsifiers and/or one or more monoethers, diethers, polyethers of polyols as W/O emulsifiers and/or one or more dimethicome copolyols as W/O emulsifiers and/or one or more fatty alcohols or fatty acids as W/O emulsifiers and/or one or more sorbitan esters as W/O emulsifiers and/or one or more methyl glucose esters as W/O emulsifiers and also comprising if desired one or more O/W emulsifiers, obtained in such a way that a mixture of basic components comprising an aqueous phase, an oil phase, one or more inventive emulsifiers is produced, being the HLB value of the emulsifier or emulsifier combination located between 2 and 14.

US2006/00165739 discloses a composition for comprising an alcohol-free microemulsion comprising a surfactant and a lipophilic and/or a hydrophilic linker for cosmetic or hair applications. These compositions have the ability to microemulsify sebum and to enhance the penetration of skin or hair active ingredients. This document discloses both hydrophilic and lipophilic linkers and a laundry list of active ingredients such as vitamins, minerals, humectants, emollients, anti-oxidants, oils, lipids, botanicals, tanning compounds, skin lightening compounds, UVA absorbers, UVB absorbers, sunscreens, infrared reflectors and infrared absorbers.

Further, there are various research publications about hydrophilic and lipophilic linkers in surfactants mixtures, enhancing solubilisation in microemulsion etc, however, these publications do not disclose that specific linkers that may be used to solubilise beneficial oils such as mineral oil in wormlike micelles in a surfactant system.

Therefore, there is still a need to solubilise beneficial oils such as mineral oil in wormlike micelles in surfactant systems.

It is therefore an object of the present invention to provide a personal wash composition comprising functional levels of beneficial oil such as mineral oil.

It is another object of the present invention to solubilise mineral oil in wormlike micelles in a surfactant system.

It is yet another object of the present invention to provide a personal wash composition that is thermodynamically stable.

It is yet another object of the present invention to deposit mineral oil to a substrate.

It is yet another object of the present invention to provide good sensory benefits to the substrate.

Surprisingly, it has been found that mineral oil can be solubilised in wormlike micelles in a surfactant system with specific hydrophilic or lipophilic linker molecules.

### SUMMARY OF THE INVENTION

Accordingly, in a first aspect the invention provides a personal wash composition comprising wormlike micelles comprising of 0.1-10% of an oil composition comprising mineral oil and a hydrophilic or lipophilic linker molecule, 10-50% w of a surfactant, 1-25% w of a water soluble electrolyte and water.

In a second aspect, the invention provides the use of the composition according to invention for treatment of hair.

In a third aspect the invention provides a method of treating a substrate comprising the steps in sequence of applying onto a substrate a composition comprising wormlike micelles comprising of 0.1-10% of an oil composition comprising mineral oil and a hydrophilic or lipophilic linker molecule, 10-50% w of a surfactant, 1-25% w of a water soluble electrolyte and water, allowing the substrate to be in contact with the composition for at least 1 min and rinsing the substrate with water.

In the context of the present invention, the reference to “substrate” typically means skin or scalp or hair fibre.

These and other aspects, features and advantages will become apparent to those of ordinary skill in the art from a reading of the following detailed description and the appended claims. For the avoidance of doubt, any feature of one aspect of the present invention may be utilised in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

### DETAILED DESCRIPTION OF THE INVENTION

In a first aspect, the invention provides a personal wash composition comprising wormlike micelles comprising of an oil composition of mineral oil and a hydrophilic or lipophilic linker molecule, a surfactant, a water soluble electrolyte and water.

### Oil Composition

The personal wash composition of the invention comprises an oil composition. The oil composition comprises a mineral oil and a lipophilic or hydrophilic linker molecule.

### Mineral Oil

Mineral oil used in the present invention is any of various colorless, odorless, light mixtures of alkanes in the C₁₀ to C₄₀ range from a non-vegetable (mineral) source, particularly a distillate of petroleum. Other names, similarly include white oil (white oils are highly refined mineral oils that consist of saturated aliphatic and alicyclic nonpolar hydrocarbons), liquid paraffin, and liquid petroleum. There are three basic classes of refined mineral oils: paraffinic oils, based on n-alkanes or sometimes branched, naphthenic oils, based on cycloalkanes and aromatic oils, based on aromatic hydrocarbons. These oils have a density of around 0.8 g/cm³.

The preferred oils are white mineral oils having a viscosity ranging from 3 cSt to 240 cSt. The unit cSt (centi stokes) is a commonly used unit for viscosities of mineral oils, and is more widely used than the SI unit, however, for the sake of completeness, 1 cSt equals 1 m²/s. They are sold under trade name Hydrobrite, Kaydol, Parol, Dosetol, Protol, Rudol, Blandol, Carnation, Medinol, Klearol, Lytol by Sonneborn. LLPO (light liquid paraffin oil) having alkyl chain length between 10-16 is also preferred.

### Linker Molecule

The oil composition of the present invention comprises a linker molecule. The linker molecule used herein may be a hydrophobic (lipophilic) linker molecule with <1% solubility in water, or a hydrophilic linker molecule.

Linker molecules are chemical additives used in surfactant systems that enhance the surfactant-oil (lipophilic) or surfactant-water (hydrophilic) interactions. However, in the composition of the present invention linkers that enhance surfactant-oil interactions are preferred.

Without wishing to be bound by theory, it is thought that the lipophilic linkers exhibit an orientation at the interface (in oil-water-surfactant system) due to a small polar group and a large hydrophobic tail. Since larger part of these lipophilic linkers will be in the oil phase, oil molecules lying next to this will be ordered. This enhanced ordering results in an enhanced interaction between the oil molecules and the surfactant and the oil molecules. Therefore, if the linker molecules have <1% of solubility in water, they will predominantly stay in oil phase and will be present at the oil-water interface for the formation of worm like micelles. In absence of these specific lipophilic linker molecules having <1% of solubility in water, the micelle formation or phase inversion conditions are not possible to achieve.

Some hydrophilic linkers, as listed herein below, are also considered within the scope of the present invention.

The linker molecule is preferably selected from saturated mono- or di-carboxylic acid (preferably capryllic acid, lauric acid, azelaic acid and/or sebacic acid), mono- di- or tri-alcohol (preferably dodecanol, dodecane diol, and/or mono propylene glycol), mono hydroxy-di-carboxylic acid (preferably mallic acid), mono hydroxy-tri-carboxylic acid (preferably citric acid), C₈-C₂₂ alkyl ethoxy alcohol (preferably C₈-C₁₈ alkyl ethoxy alcohol, more preferably C₈-C₁₅ alkyl ethoxy alcohol, still more preferably C₈-C₁₂ alkyl ethoxy alcohol; wherein the alkyl ethoxy alcohol preferably comprises 1 to 5 moles of ethylene oxide per mole of alcohol, preferably 1 to 4 moles of ethylene oxide per mole of alcohol, more preferable 2 to 3 moles of ethylene oxide per mole of alcohol), C₁-C₃ alkyl esters of fatty acids (preferably methyl laurate), Carboxyl amino acid (preferably glutamic acid) and mixtures thereof.

Alternatively the linker molecule may be selected from benzene-dicarboxylic acids, preferably benzene-1,2-dicarboxylic acid (phthalic acid), dimethoxybenzene, preferably 1,3-dimethoxybenzene, or mixtures thereof.

Combinations of these linker molecules are also contemplated.

The oil composition is present in the personal wash composition in a concentration of 0.1-10%, preferably not less than 1%, more preferably not less than 3% but typically not more than 8%, preferably not more than 7%, more preferably not more than 6% weight of the total composition.

The oil composition preferably comprises the mineral oil and the linker molecule in a ratio of between 9:1 (9 parts of oil to 1 part of linker) to 1:1 (1 part of oil to 1 part of linker).

### Surfactant

The personal wash composition of the invention comprises a surfactant or a mixture of surfactants. Surfactants are included in the composition for primary cleaning action.

Any surfactant known in the art for use in personal wash compositions may be used herein.

Suitable anionic surfactants are the alkyl sulphates, alkyl ether sulphates, alkaryl sulphonates, alkyl succinates, alkyl sulphosuccinates, acyl taurates, acyl glutamates, N-alkoyl sarcosinates, alkyl phosphates, alkyl ether phosphates, alkyl ether carboxylates, and alpha-olefin sulphonates, especially their sodium, potassium, magnesium, ammonium and mono-, di- and triethanolamine salts. The alkyl and acyl groups generally contain from 8 to 18 carbon atoms and may be unsaturated. The alkyl ether sulphates, alkyl ether phosphates and alkyl ether carboxylates may contain from 1 to 10 ethylene oxide or propylene oxide units per molecule, and preferably contain from 1 to 5 ethylene oxide units per molecule, more preferably contain 2 to 3 ethylene oxide units per molecule.

Examples of suitable anionic surfactants include are sodium lauryl ether sulphate, sodium oleyl succinate, ammonium lauryl sulphosuccinate, ammonium lauryl sulphate, sodium dodecylbenzene sulphonate, triethanolamine and sodium salts of dodecylbenzene sulphonate and sodium N-lauryl sarcosinate.

The most preferred anionic surfactants are sodium lauryl ether sulphate 1 EO, 2 EO, and 3 EO, ammonium lauryl sulphate, ammonium lauryl ether sulphate 1 EO, 2 EO and 3 EO, and triethanolamine and sodium salts of dodecylbenzene sulphonate. Sodium lauryl ether sulphate 3 EO is particularly preferred as it gives a clear and stable shampoo when used at high viscosity.

Suitable cationic surfactants are quaternary ammonium salts according to the present invention are quaternary ammonium salts characterised in that the ammonium salt has the general formula: R₁R₂R₃R₄N⁺X⁻, wherein R₁ and R are independently selected from C₁-C₁₈ alkyl group, while R₃ and R₄ are independently selected from is a C₁-C₃ alkyl group and X is an inorganic anion. R₁ is preferably a C₈-C₁₆ straight chain alkyl group. R₂-R₄ are preferably methyl groups. The inorganic anion is preferably chosen from halide, sulphate, bisulphate or OH⁻. Thus, for the purposes of this invention, a quaternary ammonium hydroxide is considered to be a quaternary ammonium salt.

More preferably the anion is a halide ion or hydroxide, still more preferably a chloride or hydroxide. In case alkalinity is required, the anion is preferably hydroxide. Cetyl-trimethylammonium chloride is a specific example of a suitable compound and commercially abundantly available. Other suitable cationic surfactants may include tetramethylammonium hydroxide or chloride, octyltrimethylammonium hydroxide or chloride, dodecyltrimethylammonium hydroxide or chloride, hexadecyltrimethyl-ammonium hydroxide or chloride, octyldimethylbenzylammonium hydroxide or chloride, decyldimethylbenxylammonium hydroxide or chloride, didodecyldimethylammonium hydroxide or chloride, dioctadecyldimethylammonium hydroxide or chloride, tallow trimethylammonium hydroxide or chloride, cocotrimethylammonium hydroxide or chloride.

Suitable nonionic surfactants may include condensation products of aliphatic (C₉-C₁₈) primary or secondary linear or branched chain alcohols or phenols with alkylene oxides, usually ethylene oxide and generally having from 2 to 30 ethylene oxide (EO) groups, more preferably at least 3 EO, still more preferably at least 5 EO, but usually not more than 25 EO, more preferably not more than 20 EO or even not more than 15 EO.

Other suitable nonionics include alkylpolyglycosides and mono- or di-alkyl alkanolamides. Examples of the latter nonionics include coco mono- or di-ethanolamide and coco mono-isopropanolamide.

Suitable amphoteric and zwitterionic surfactants may include alkyl amine oxides, alkyl betaines, alkyl amidopropyl betaines, alkyl sulphobetaines (sultaines), alkyl glycinates, alkyl carboxyglycinates, alkyl amphopropionates, alkylamphoglycinates, alkyl amidopropyl and hydroxysultaines, wherein the alkyl and acyl groups gave 8 to 19 carbon atoms. Examples include lauryl amine oxide, cocodimethyl sulphopropyl betaine and preferably lauryl betaine, cocamidopropyl betaine and sodium cocamphopripionate.

Examples of most preferred surfactants include sodium lauryl ether sulphate 1 EO, 2 EO, and 3 EO, ammonium lauryl sulphate, cocamidopropyl betaines.

The surfactant is present in the personal wash composition in a concentration of 6-50%, preferably at least 10%, more preferably at least 15%, still more preferably at least 20%, even more preferably at least 25% but typically not more than 45%, preferably not more than 40%, more preferably not more than 35%, still more preferably not more than 30% by weight of the composition.

### Water Soluble Electrolyte

The personal wash composition of the invention comprises an electrolyte. The electrolyte used in the present invention is a water soluble electrolyte. Electrolytes are used to obtain the single phase, which is dependent on surfactant and oil, as well as to achieve viscosity in the formulation.

Chloride salts such as sodium chloride, potassium chloride, calcium chloride, magnesium chloride, zinc chloride, ferric chloride, aluminium chloride, sulphate salts such as sodium sulphate, magnesium sulphate etc.

Most preferred electrolytes are chlorides such as sodium chloride and potassium chloride and magnesium sulphate.

The water soluble electrolyte is present in the personal wash composition in a concentration of 1-25%, preferably at least 3%, more preferably at least 5%, still more preferably at least 10%, even more preferably at least 15% but typically not more than 23%, preferably not more than 20%, more preferably not more than 18% by weight of the composition.

### Water

The personal wash compositions of the invention are preferably aqueous based, water forming the basis of the continuous phase of the emulsion.

The composition comprises water adding up to a 100% by weight of the total composition. The composition preferably comprises water in an amount of between 50 and 90%, more preferably not more than 85%, still more preferably not more than 83% but typically not less than 55%, preferably not less than 60%, or more preferably not less than 65% by weight of the composition.

### Product Format

The compositions of the invention are preferably rinse-off compositions, i.e., suitable for applying to the hair, left thereon for an appropriate period of time and then rinsed off with water.

Compositions in accordance with the present invention may be optically clear.

### Other Ingredients

Depending upon the type of personal wash, one or more additional ingredients conventionally incorporated into shampoo formulations may be included in the compositions of the invention. Such additional ingredients include antibacterial agents, antidandruff agents, foam boosters, perfumes, colouring agents, preservatives, viscosity modifiers, opacifiers, pearlescers, antibacterial agents, antidandruff agents, proteins, polymers, buffering or pH adjusting agents, foam boosters, moisturising agents, herb or other plant extracts and other natural ingredients.

The composition according to the invention may further comprise a silicon oil for improved hair conditioning and sensory benefits. Silicon oil may be used in neat form or in the form of an emulsion.

When present, silicon oil is present in the personal wash composition in a concentration of 0.1 to 10%, preferably at least 0.5%, more preferably at least 1%, still more preferably at least 2%, even more preferably at least 3% but typically not more than 9%, preferably not more than 7%, more preferably not more than 6%, still more preferably not more than 5%, even more preferably not more than 4% by weight of the composition.

### Viscosity

The composition is preferably somewhat viscous. Consumers typically do not associate water thin compositions with high active (i.e. concentrated) detergent compositions.

Viscosity describes a fluid's internal resistance to flow and may be thought of as a measure of fluid friction. Simply put, the less viscous the fluid is, the greater its ease of movement (fluidity).

The Viscosity of the compositions according to the invention is preferably between 0.2 and 40 Pa·s (25° C. and 20 s⁻¹), more preferably not less than 0.5, still more preferably not less than 2, even more preferably not less than 5 but typically not more than 30, still more preferably not more than 20 or even more than 10 Pa·s, when measured with a TA instrument rheometer AR-1000, with a cone and plate set-up, acrylic/steel 4 cm diameter, 2° angle, truncation gap 52-58 micrometer, in steady flow operation.

### Use of the Composition

In a second aspect, the invention provides the use of the composition according to the invention for treatment of hair.

### Method of Treating a Substrate

In a third aspect, the invention provides a method of treating a substrate comprising the steps in sequence of applying onto a substrate a composition comprising wormlike micelles comprising of 0.1-10% of an oil composition comprising mineral oil and a hydrophilic or lipophilic linker molecule, 10-50% w of a surfactant, 1-25% w of a water soluble electrolyte and water, allowing the substrate to be in contact with the composition for at least 1 min and rinsing the substrate with water.

The invention will now be illustrated by means of the following non-limiting examples.

### EXAMPLES

### Materials

### Linker Molecules

| INCI name | Trade name | Manufacturer |
|---|---|---|
| Octanoic acid | Caprylic Acid | Sigma Aldrich |
| Do decanoic Acid | Lauric acid | Sigma Aldrich |
| Oleic acid | Oleic acid | Sigma Aldrich |
| Span 80 | Span 80 | Sigma Aldrich |
| Tween 60 | Tween 60 | Sigma Aldrich |
| APG 425 | Glucocopon 425 N/HH | BASF |
| Phthalic acid | Phthalic acid | Sigma Aldrich |
| Azelaic acid | Azelaic acid | Sigma Aldrich |
| 1,12 Dodecane diol | 1,12 Dodecane diol | Sigma Aldrich |
| Citric acid | Citric acid | Merck |

### Other Materials

| INCI name | Trade name | Manufacturer |
|---|---|---|
| Sodium Laureth | LES 170/Texapon | Galaxy/Cognis/ |
| Sulfate | N701 | Hunsman |
| Cocoamidopropyl | CAPB SB/Dehyton | Galaxy/Cognis/ |
| Betaine | KE/Tegobetaine | Hunsman/Goldschmidt |
| | CK KF 400 | |
| Citric Acid | Citric Acid | Merck |
| | (anhydrous) | |
| Carbomer | Carbopol 980 | Goodrich |
| Pearliser - Glycol | Euperlan KE 4515 | Cognis |
| Distearate and Sodium | | |
| Laureth Sulphate, | | |
| Cocamidopropyl Betaine | | |
| Silicone Oil | Wacker 9815 | Dow Corning |
| Parfum | Saturne P | Givaudan |
| Sodium Chloride | NaCl | Aldrich |
| Sodium Hydroxide | Sodium Hydroxide | Sigma Aldrich |
| Guar | BF Jaguar C14 | Rhodia |
| Hydroxypropyltrimonium | | |
| Chloride | | |
| Guar | BF Jaguar C17 | Lamberti |
| Hydroxypropyltrimonium | | |
| Chloride | | |
| DMDM Hydantoin | Glydant | Lonza |
| Methylchloroiso- | Kathon CG | Rohm&Haas |
| thiazolinone and | | |
| Methylisothiazolinone | | |
| Disodium EDTA | Versene NA2 | Dow Chemicals |
| | Dihydrate | |
| Polypropylene Glycol | Polypropylene Glycol | Sigma Aldrich |
| P400 | | |
| Sodium Hydroxide | Sodium Hydroxide | Merck |
| Mineral Oil | Lytol | Sonneborne |
| Aqua | Water | Lab |

### Example 1

### Effect of the Linkers on the Phase of the Composition

In this example, the effect of different linker molecules is compared. Ex 1 and Ex 2 are compositions according to the present invention which are compared to Comp A (Comparative composition) comprising a linker molecule outside the scope of the present invention.

### Preparing the Compositions:

A mixture of surfactants as mentioned in the below table were mixed with water. To this solution, a mixture of linker and mineral oil was added. The requisite amount of sodium chloride (electrolyte) was also added to this mixture. The whole solution was vortexed nearly 15 minutes and then kept in a water bath at 50±1° C. for 24 hours. The compositions were visually checked for any phase separation. The micro structure of the formulation was characterised by Rheological measurements.

### Rheological Measurements:

A TA instrument, AR-1000 series, controlled stress rheometer was used to carry out rheological measurements. For generating flow curves (viscosity vs. shear rate), a cone and plate geometry having a diameter of 4 cm and cone angle of 2° was used. The gap between the cone and plate was set to be 58 m. Same cone and plate geometry had been used in oscillatory rheology experiments. The data was analyzed using TA data analysis software. All the experiments had been performed at 25° C. temperature. Evaporative loss had been prevented by using solvent trap on the sample. Before every rheological measurement, the samples were subjected to high speed centrifugation (Super spin RV-FA), with 9000 rpm for 20 min to avoid any air bubble, trapped inside it.

| | Surfactant | | | | | | | |
|---|---|---|---|---|---|---|---|---|
| | w % | | | | | | | |
| | (LES 170 + | Mineral | | | | | | Type of |
| | CAPB | oil | Linker | | Electrolyte | Water | | micro |
| Set | SB) | w % | Type | w % | w % | w % | Phase | structure |
| Ex 1 | 14.43 | 2.70 | Caprylic | 0.30 | 4.50 | 78.25 | 1- | Worm |
| | | | acid | | | | phase | like |
| | | | | | | | | Micelle |
| Ex2 | 14.43 | 2.70 | Lauric | 0.30 | 4.50 | 78.25 | 1- | Worm |
| | | | acid | | | | phase | like |
| | | | | | | | | Micelle |
| Comp A | 14.43 | 2.70 | Oleic | 0.30 | 4.50 | 78.25 | 2- | |
| | | | acid | | | | phase | |

The table above shows that the saturated fatty acid linkers according to the invention provide the worm like micelles in a single phase, while a non-saturated fatty acid linker gives phase separation.

### Example 2

### Effect of Commonly Used Linkers on the Phase of the Composition

In this example, the effect of commonly used linkers is compared to different linker molecules according to the present invention. Comp B to D are comparative compositions comprising surfactants used as common linker molecules which are compared to Ex 3 to Ex 6, compositions comprising linker molecules according to the present invention in different concentrations.

### Preparing the Compositions:

A mixture of surfactants as mentioned in the below table were mixed with water. To this solution, a mixture of linker and mineral oil was added. The requisite amount of sodium chloride (electrolyte) was also added to this mixture. The whole solution was vortexed nearly 15 minutes and then kept in a water bath at 50±1° C. for 24 hours. The compositions were visually checked for any phase separation. The micro structure of the formulation was characterised by Rheological measurements.

Rheological measurements were recorded as detailed in Example 1.

| | Surfactant | Mineral | Linker | | Electrolyte | Water | | Type of |
|---|---|---|---|---|---|---|---|---|
| Set | w % | oil w % | Type | w % | w % | w % | Phase | microstructure |
| Comp B | 14.43 | 2.70 | Span | 0.30 | 4.50 | 78.25 | 2- | |
| | | | 80 | | | | phase | |
| Comp C | 14.43 | 2.70 | Tween | 0.30 | 4.50 | 78.25 | 2- | |
| | | | 60 | | | | phase | |
| Comp D | 14.43 | 2.70 | APG | 0.30 | 4.50 | 78.25 | 2- | |
| | | | 425 | | | | phase | |
| | | | NHH | | | | | |
| Ex3 | 15.21 | 2.0 | Phthalic | 0.3 | 10 | 72.49 | 1- | Worm |
| | | | acid | | | | phase | like |
| | | | | | | | | micelle |
| Ex4 | 15.21 | 3.0 | Azelaic | 0.1 | 6.6 | 75.09 | 1- | Worm |
| | | | acid | | | | phase | like |
| | | | | | | | | micelle |
| Ex5 | 15.21 | 4.12 | 1,12 | 0.56 | 10.05 | 70.06 | 1- | Worm |
| | | | Dodecanediol | | | | phase | like |
| | | | | | | | | micelle |
| Ex6 | 15.21 | 4.12 | Citric | 0.56 | 10.05 | 70.06 | 1- | Worm |
| | | | acid | | | | phase | like |
| | | | | | | | | micelle |

The table above shows that the linker molecules according to the invention provide a stable composition with worm micelles, while common surfactants as used in the art give phase separation.

### Example 3

### Deposition of Mineral Oil on Hair

In this example, the deposition of mineral oil on hair is measured post treating with the compositions according to the invention (Ex 7 and Ex 8) and compared to a control composition (Comp E) without mineral oil.

### Preparing the Compositions:

In a beaker, the requisite amount of citric acid was added to water till the pH of the solution was 2-3. Carbopol was slowly added to this solution under constant stirring (˜600 rpm). This carbopol slurry was added to 25% SLES solution. To this solution, a Pearliser was added and mixed thoroughly. In a separate container Jaguar C14, Jaguar C17, perfume, mineral oil and linker was mixed. This mixture was then added to the carbopol mixture. This was followed by the addition of silicone emulsion and CapB, sequentially. The ingredients were allowed to be mixed for 10 min. Further, Glydant LTD and Kathon CG were added with constant stirring, followed by EDTA and NaCl addition. Finally, the pH of the mixture was adjusted to between 5-7 using NaOH solution.

### Switch Preparation and Shampoo Application:

For each formulation in a test, 3× bundles of 5×2.5 g, 6″ Dark Brown European hair switches were used. The switches were washed as bundles of five, first with 14% sodium lauryl ether sulphate solution (base wash) as follows. The bundles of 5 switches were wetted together under tap water at a flow rate of 4 l/minute for 5 seconds. 1.25 g base wash was applied from a syringe and agitated with gloved fingers for 30 seconds followed by rinsing for 30 seconds under tap water. Another 1.25 g base wash was applied to the other side of the switch bundle and again agitated for 30 seconds followed by rinsing for 30 seconds under tap water. Excess water was removed from the switch by moving your index finger and forefinger along the length of the switch. This wash process was then repeated with the test shampoo instead of the base wash.

### Measurement of Deposition:

The hair switches were dried overnight, in ambient conditions. The hair from the switches were cut (from the very edge of the metal clip) to smaller dimensions of about 1-2 cm length and were immersed in about 150 ml of hexane in a glass beaker. This was then transferred to an ultrasonic water bath kept at an elevated temperature of 45±2° C. The mineral oil was extracted in hexane for 15 min. The process of extraction was repeated twice and all the extracts were pooled together. The extract was then transferred to an RB flask and hexane was evaporated to a 2-4 ml volume and then dried by a flush of N₂ gas. The residue was then re-dissolved using hexane and analyzed with Gas chromatography (GC). The amount of mineral oil in the extract was calculated from the standard calibration curve and is reported as micro gram (ug) of mineral oil per gram of hair.

| | | Control | 2% MO shampoo | 3% MO shampoo |
|---|---|---|---|---|
| Set | | (Comp E) | (Ex 7) | (Ex 8) |
| Water | | 73.89 | 71.88 | 69.04 |
| SLES 1 EO | | 13.62 | 13.62 | 13.62 |
| Citric acid | | 0.02 | 0.02 | 0.03 |
| Carbopol 980 | | 0.42 | 0.42 | 0.4 |
| Euperlan KE 4515 | | 2.62 | 2.62 | 2.5 |
| Linker | Type | — | Caprylic acid | Azelaic acid |
| | w % | 0 | 0.2 | 0.1 |

| | | | |
|---|---|---|---|
| Mineral oil | 0 | 1.8 | 3 |
| Perfume | 0.9 | 0.9 | 0.9 |
| Jaguar C14 | 0.15 | 0.15 | 0.15 |
| Jaguar C17 | 0.05 | 0.05 | 0.05 |
| DC 5-7051 HS or | 1.65 | 1.65 | 1.5 |
| Wacker 9815 | | | |
| Tegobetaine CK | 1.6 | 1.6 | 1.6 |
| Glydant LTD | 0.1 | 0.1 | 0.1 |
| (55%) | | | |
| Kathon CG | 0.06 | 0.06 | 0.06 |
| EDTA | 0.1 | 0.1 | 0.1 |
| NaCl | 4.6 | 4.6 | 6.6 |
| NaOH solution | 0.23 | 0.23 | 0.25 |
| Deposition (ug/g) | 74.4 | 121.8 | 207 |

The above table shows enhanced deposition of mineral oil on the hair with compositions according to the invention than with the control formulation.

### Example 4

### Sensory Comparison

In this example, the composition according to the invention (Ex 9) is compared to a control product on various sensory aspects.

Set

| | Composition | | |
|---|---|---|---|
| | Same as Ex 9 but devoid of mineral oil and linker | | |
| Control | (2%) and water adjusted to a 100% accordingly. | | |
| Ex 9 | Ingredients | Type | Concentration w % |
| | Surfactant | SLES/CAPB | 14/1.6% |
| | Mineral oil | Lytol | 1.8% |
| | Linker | Caprylic acid | 0.2% |
| | Electrolyte | NaCl | 4.6% |
| | Water | Deionised water | 75.6% |
| | Guar | Boron Free | 0.15% |
| | Hydroxypropyl- | Jaguar C13 | |
| | trimonium | | |
| | Chloride | | |
| | Guar | Boron Free | 0.05% |
| | Hydroxypropyl- | Jaguar C17 | |
| | trimonium | | |
| | Chloride | | |
| | Silicon oil | DC 1788 | 2% |

The sensory test was carried out using standard salon half head protocol, wherein the control shampoo was applied on half of the head and the experimental shampoo was applied on the other half. The shampoo application, cleaning procedure and further blow drying was performed by a hair stylist. The data shared in the table is the average response from 36 female panelists having dry/damaged/dry-damaged hair types. The dry sensory effects are given in the table below.

| Set | Control | Ex 9 |
|---|---|---|
| Ease of styling | 13 | 21 |
| Better control | 10 | 21 |
| Ease of combing | 13 | 20 |
| Slippery feel | 13 | 20 |
| Smooth feel | 13 | 22 |
| More softness | 10 | 22 |

The results in the table above show that the composition according to the invention is better on all sensory aspects than the control sample.

The wet sensory effects are given in the table below.

| Set | Control | Ex 9 |
|---|---|---|
| Time to rinse | 12 | 16 |
| Alignment during rinse | 15 | 17 |
| Ease of wet combing - first stroke | 13 | 19 |
| Slippery feel | 10 | 23 |
| Residual/coating | 13 | 18 |

The results in the table above show that the composition according to the invention is better on all sensory aspects than the control sample.

### Example 5

### Effect of the Composition on Hair Alignment

In this example, hair alignment after using different compositions was compared.

### Preparing the Compositions:

In a beaker, the requisite amount of citric acid was added to water till the pH of the solution was 2-3. Carbopol was slowly added to this solution under constant stirring (˜600 rpm). This carbopol slurry was added to 25% SLES solution. To this solution, a Pearliser was added and mixed thoroughly. In a separate container Jaguar C14, Jaguar C17, perfume, mineral oil and linker was mixed. This mixture was then added to the carbopol mixture. This was followed by the addition of silicone emulsion and CapB, sequentially. The ingredients were allowed to be mixed for 10 min. Further, Glydant LTD and Kathon CG were added with constant stirring, followed by EDTA and NaCl addition. Finally, the pH of the mixture was adjusted to between 5-7 using NaOH solution.

Switch preparation and shampoo application was carried out as explained in Example 3.

Post shampoo application, the switches were allowed to hang on a stand and were combed through twice using the wide-toothed end to remove tangles and then twice with the other end of the comb to align the fibres. After the final comb, the switches were followed with the fingers and thumb to remove excess water. A new comb was used for each product. The switches were then hung to dry using the bulldog clip, in the drying cabinet (50 Celcius) overnight. After drying, the switches were removed, attached to the stands and placed in an environment controlled room to acclimatise for at least 2 hours. Images were captured in a controlled environment with fixed lighting conditions using a DigiEye Image Capture Box from Verivide and using a Nikon D70 camera with Nikkon 60 mm lens. Once captured, they were then presented to expert panelists in order for them to rank using a line scale approach of 1-10. Statistical methods were then applied to the test data to establish whether the panelists were able to detect statistically significant differences between the alignment states of hair bundles treated with different products. The R index value obtained from the statistical method is given in table to show the performance.

The compositions are specified in the table below.

| Set* | | Control | MO only | MO + S | S only |
|---|---|---|---|---|---|
| Compo- | SLES | 18.25 | 18.25 | 18.25 | 18.25 |
| sition | Citric acid | 0 | 0.04 | 0.04 | 0.04 |
| | Carbopol980 | 0 | 0.4 | 0.4 | 0.4 |
| | Silicone9815 | 0 | 0 | 1.11 | 1.11 |
| | Euperlan | 0 | 2.4 | 2.4 | 2.4 |
| | Perfume | 0.9 | 0.8 | 0.8 | 0.8 |
| | Mineral | 0 | 0.5 | 0.5 | 0 |
| | Oil(9:1 ratio | | | | |
| | of mineral | | | | |
| | oil:linker) | | | | |
| | CAPB | 5.3 | 5.3 | 5.3 | 5.3 |
| | NaCl | 1 | 2 | 2.86 | 0.8 |
| | JagC17S | 0.05 | 0 | 0 | 0 |
| | JagC13S | 0.15 | 0.2 | 0.2 | 0.2 |

| | | | | |
|---|---|---|---|---|
| Hair alignment (R Index) | 0.5 | 0.57 | 0.85 | 0.67 |
| Phase | 1-phase | 1-phase | 1-phase | 1-phase |
| Microstructure | Worm like | Worm like | Worm like | Worm like |
| | micelle | micelle | micelle | micelle |

MO = mineral oil,
S = silicon oil

The results in the table above shows that mineral oil gives already about the same effect in hair alignment (conditioning) as silicon oils. Additionally, it is surprisingly found that the combination of mineral oil and silicon oil gives an even better hair alignment than compositions outside the scope of the invention.

### Example 6

### Effect of the Composition on Smoothness, Softness and Ease of Combing

In this example, different sensory aspects like smoothness, softness and ease of combing after using different compositions were compared.

The same compositions were used as in example 5.

Switch preparation and shampoo application was carried out as explained in example 3.

Post shampoo application, the switches were allowed to hang on a stand and were combed through twice using the wide-toothed end to remove tangles and then twice with the other end of the comb to align the fibres. After the final comb, the switches were followed with the fingers and thumb to remove excess water. A new comb was used for each product. The switches were then hung to dry using the bulldog clip, in the drying cabinet (50 Celcius) overnight. After drying, the switches were removed, attached to the stands and placed in an environment controlled room to acclimatise for at least 2 hours. Images were captured in a controlled environment with fixed lighting conditions using a DigiEye Image Capture Box from Verivide and using a Nikon D70 camera with Nikkon 60 mm lens. Once captured, they were then presented to expert panelists in order for them to rank using a line scale approach of 1-10. Statistical methods were then applied to the test data to establish whether the panelists were able to detect statistically significant differences between the alignment states of hair bundles treated with different products. The R index value obtained from the statistical method is given in table to show the performance.

| | Sensorial | | | | |
|---|---|---|---|---|---|
| | Smooth- | Soft- | Ease of | | |
| Set | ness | ness | combing | Phase | Microstructure |
| Control | 0.5 | 0.5 | 0.5 | 1-phase | Worm like micelle |
| MO only | 0.6 | 0.59 | 0.51 | 1-phase | Worm like micelle |
| MO + S | 0.97 | 0.96 | 0.99 | 1-phase | Worm like micelle |
| S only | 0.8 | 0.82 | 0.95 | 1-phase | Worm like micelle |

MO = mineral oil,
S = silicon oil

The results in the table above shows that mineral oil as such also provides conditioning benefits. Additionally it is shown that the combination of mineral oil and silicon oil gives an even better smoothness, softness and ease of combing than compositions outside the scope of the invention.

1. A personal wash composition comprising wormlike micelles comprising
a 0.1-10% of an oil composition comprising
i Mineral oil; and
ii A hydrophilic or lipophilic linker molecule;
b 6-50% w of a surfactant;
c 1-25% w of a water soluble electrolyte; and
d Water
wherein the linker molecule is selected from:
i saturated mono- or di-carboxylic acid, mono- di- or tri-alcohol, mono hydroxy-di-carboxylic acid, mono hydroxy-tri-carboxylic acid, C₈-C₂₂ alkyl ethoxy alcohol, C₁-C₃ alkyl esters of fatty acids, carboxyl amino acid and mixtures thereof; or
ii benzene-dicarboxylic acids, dimethoxybenzene, or mixtures thereof; or
iii mixtures thereof.

2. A composition according to claim 1 wherein the oil composition comprises the mineral oil and the linker molecule in a ratio of between 9:1 and 1:1.

3. A composition according to claim 1 wherein the surfactant is selected from hard water tolerant anionic and/or betaines.

4. Use of the composition according to claim 1 for treatment of hair.

5. A method of treating a substrate comprising the steps in sequence of:
a applying onto a substrate a composition comprising wormlike micelles comprising:
i 0.1-10% of an oil composition comprising:
(1) Mineral oil; and
(2) a hydrophilic or lipophilic linker molecule;
ii 6-50% w of a surfactant;
iii 1-25% w of a water soluble electrolyte; and
iv Water;
wherein the linker molecule is selected from:
i saturated mono- or di-carboxylic acid, mono- di- or tri-alcohol, mono hydroxy-di-carboxylic acid, mono hydroxy-tri-carboxylic acid, C₈-C₂₂ alkyl ethoxy alcohol, C₁-C₃ alkyl esters of fatty acids, carboxyl amino acid and mixtures thereof; or
ii benzene-dicarboxylic acids, dimethoxybenzene, or mixtures thereof; or
iii mixtures thereof.
b allowing the substrate to be in contact with the composition for at least 1 min; and
c rinsing the substrate with water.
